(19) United States
(12) Patent Application Publication
(10) Pub. No.: US 2017/0311998 A1
(43) Pub. Date: Nov. 2, 2017

(54) DEVICE FOR APPLYING BONE REPLACEMENT PASTES

(71) Applicant: Mathys AG Bettlach, Bettlach (CH)

(72) Inventors: Yvonne HUG, Zofingen (CH); Björn OLBRICHT, Biel (CH)

(21) Appl. No.: 15/520,696

(22) PCT Filed: Oct. 22, 2015
(86) PCT No.: PCT/CH2015/000155

§ 371 Date: Apr. 20, 2017

(30) Foreign Application Priority Data
Oct. 27, 2014 (DE) 20 2014 008 486.6

(51) Int. Cl. A61B 17/88

(52) U.S. Cl. CPC A61B 17/8805; A61B 17/8816; A61B 17/8822

(57) ABSTRACT

Device (1) for applying bone replacement pastes or bone cements, wherein the device (1) comprises: A) a container-like base part (4) with a first end (5), a second end (6) and a cavity (7), which defines a first longitudinal axis (3), is closed at the first end (5) and is open at the second end (6); and B) a filling piston (8) which can be inserted slidably into the cavity (7) and has a second longitudinal axis (10) and two or more hollow spaces (9) which extend through the filling piston (8) coaxially or parallel to the second longitudinal axis (10) and into which the cannula (13) of the applicator (2) can optionally be inserted.

The invention relates to a device for applying bone replacement pastes or bone cements according to the preamble of claim 1, with a kit for applying bone replacement pastes or bone cements according to the preamble of claim 37 and with a method for intraoperative preparation of a bone replacement paste and filling applicator devices according to the preamble of claim 40.

Vertebroplasty is an effective treatment option, especially for osteoporotic vertebral fractures and for the augmentation of vertebral bodies. Various injectable biomaterials such as polymethyl methacrylate (PMMA)—an autopolymerizing two-component system—or calcium phosphate cements—cements that cure hydraulically upon exposure to water—are known and are used together with suitable cement injection instruments.

In addition to bone cements, non-curing, non-polymerizing or non-hydraulically setting bone replacement pastes are also used in stabilized kyphoplastic defects and fusions of the intervertebral spaces in and around the intervertebral implants (interbody fusion). In general, bone defects caused by disease, accidents or surgery are treated with various bone replacement materials. In addition to autologous bone, synthetic bone replacement material offers several advantages over allogeneic or xenogeneic bone replacement material: no risk of transferring pathogenic bacteria, constant product properties, no ethical concerns. To facilitate the application of synthetic bone replacement materials in various administration forms such as granulates, pastes and cements, or to apply them in a minimally invasive manner, the use of dispensing/applicator systems with one or more applicator devices is required.

To perform such bone replacement-promoting treatments (kyphoplasty, vertebroplasty, interbody fusion, general bone defect fillings in treatment of the spinal column or orthopedics, trauma, CMF and dental treatments) a mixing device for mixing the components and at least one applicator device for applying the bone replacement paste through a cannula are needed.

A dispensing device for filling several applicator devices with a freshly mixed bone cement is known from document WO 2005/123162. This known dispensing device comprises a dispensing device in the form of a large-volume syringe and a distribution device comprise an inlet opening that can be connected to the dispensing device and, after this, a tubular cylinder with several lateral nipples, wherein each nipple can be connected to a syringe, so that the syringes attached to the nipples can be filled essentially simultaneously with a bone cement. Each nipple is connected to the tubular cylinder by a valve, so that the syringes fastened to the distribution device can be filled in succession or also virtually simultaneously. However, this known dispensing device has the drawback that the user must manually apply a high pressing force to the piston of the dispensing device when filling the syringes, since the tubular cylinder of the distribution device with the nipples for the syringes is relatively long, which causes a large pressure drop during filling of the syringes with high-viscosity bone cement, and in addition the transition between the chamber of the dispensing device and the inlet opening of the distribution device represents relatively great narrowing of the cross section, which in turn causes a large pressure drop.

An additional dispensing device for simultaneously refilling several syringes is known from US-A 2013/064033 LIDGREN ET AL. This known dispensing device likewise comprises a dispensing mechanism in the form of a large-volume syringe and a distribution device that can be attached to the outlet opening of the syringe, comprising an inlet nipple with a chamber and several outlet nipples arranged around the chamber in a star shape. The outlet nipples can each be connected to a syringe. This known dispensing device likewise has the drawback that when filling the syringes, the user must manually exert a high compressive force on the piston of the dispensing device. This is especially true since the transition from the cavity of the syringe to the outlet opening forms a severe constriction, which causes a large pressure drop especially in the case of high-viscosity substances, and a considerable further pressure drop also takes place in the distribution device due to the redirection of the bone cement.

The invention tries to provide help here. The invention is based on the task of creating a device that allows filling of applicators with high viscosity substance with a reduced application of force.

The invention accomplishes the task posed with a device for applying bone replacement pastes or bone cements that has the features of claim 1, with a kit for applying bone replacement pastes or bone cements that has the features of claim 37, and with a method for intraoperative preparation of the bone replacement paste and filling applicators, having the features of claim 40.

The advantages achieved by the invention essentially lie in the fact that thanks to the device according to the invention:
through the parallel arrangement of the cavities in the filling piston a large ratio between the sum of the cross-sectional surfaces of the hollow spaces in the filling piston and the cross-sectional surfaces in the cavity of the base part can be achieved, so that the flow rate of the highly viscous bone replacement paste into the cavities of the filling piston when the latter is inserted into the cavity in the base part filled with bone replacement paste remains low, leading to reduced pressure drop due to the wall friction of the bone replacement paste;
because of this large ratio between the total cross-sectional areas of the cavity in the filling piston and the cross-sectional area of the cavity in the base the pressure drop caused by the cross-sectional reduction during insertion of the filling piston into the cavity in the base filled with bone replacement paste is reduced, so that the injection force to be applied by the user is further reduced;
because of the reduced flow velocity of the bone replacement paste, the damage done to the particles contained in the bone replacement paste by wall friction can be decreased;
in the filling of the cannulas of the applicators with high viscosity substances, due to direct insertion of the cannulas into the hollow spaces in the filling piston, only short flow pathways occur and thus smaller pressure drops need to be overcome, so that reduced manual application of force by the user results; and
through the simultaneous withdrawal of the applicator piston when filling the applicator devices, initial sticking when inserting the applicator piston after filling the applicator devices can be avoided.

One component of a preferred bone replacement paste can be supplied as a dry powder mixture consisting of beta TCP granules and hyaluronic acid granules. If the powder mixture is mixed with a liquid, e.g., a physiologic saline solution, blood or derivative thereof, or antibiotics, the hyaluronic acid granules swell and surround the beta TCP granules. A non-curing, kneadable paste that can be used for the minimally invasive application of bone replacement material is formed.

For example, the application kit can also be used for other bone replacement pastes, demineralized bone matrices such as DBX or other ready-to-use bone replacement pastes that are supplied without applicator assistance.

Additional advantageous embodiments of the invention can be mentioned as follows:

In a special embodiment the device also has one or more applicators, each of which comprises a cannula with a lumen and an applicator piston that can be inserted into the lumen of the cannula.

In another embodiment of the device, the cavity has a cross-sectional area Q perpendicular to the first longitudinal axis, and each hollow space has a cross-sectional area q that is perpendicular to the second longitudinal axis, wherein the ratio between the sum of the cross-sectional areas q and the cross-sectional area Q is between 0.25 and 0.95, preferably between 0.35 and 0.9. Because of this large ratio between the sum of the cross-sectional area q of the hollow spaces and the cross-sectional area Q of the cavity in the base, the pressure drop caused by the change in cross-section is reduced, so that the injection force to be applied by the user is considerably decreased. It is also accomplished that the flow rate of the bone replacement paste into the hollow spaces can be kept low, so that a reduced pressure drop can be obtained as a result of the wall friction of the bone replacement paste.

In an additional embodiment of the device the filling piston has more than two hollow spaces, preferably 3 to 7 hollow spaces, which penetrate the filling piston coaxially or in parallel to the second longitudinal axis, and into which optionally the cannula of the applicator can be inserted. In this way it is possible to achieve the benefit that the ratio between the sum of the cross-sectional areas q of the hollow spaces and the cross-sectional area Q of the cavity can be increased, so that the pressure drop upon inserting the filling piston into the cavity can be further reduced.

In an additional embodiment of the device each of the hollow spaces has a volume v and the cavity has a volume V, wherein the ratio between the volume v of a hollow space and the volume V of the cavity is at least 0.05, preferably at least 0.1.

In another embodiment of the device the hollow spaces have hollow space axes parallel to the second longitudinal axis, which are arranged in the angles of a regular polygon located perpendicular to the second longitudinal axis.

In yet another embodiment of the device the cavity perpendicular to the first longitudinal axis and the filling piston on the outside perpendicular to the second longitudinal axis have a non-circular cross-sectional area, preferably a cloverleaf-shaped, oval or elliptical cross-sectional area. This provides the advantage that the ratio between the sum of the cross-sectional areas q in the case of two or three hollow spaces and the cross-sectional area Q of the cavity can be increased, so that the pressure drop upon inserting the filling piston into the cavity can be further reduced.

In an additional embodiment of the device a grip is attached to the filling piston, preferably at the back end of the filling piston. Thus when the cannula of an applicator is inserted into one of the hollow spaces of the filling piston filled with bone replacement paste, the filling piston can be pressed against the base part via the grip, so that the filling piston is not raised by the pressure on the bone replacement paste in the cavity of the base part, and the bone replacement paste present in the hollow space of the filling piston is delivered into the lumen of the cannula of the applicator.

In an additional embodiment of the device the base part has a funnel shape at the second end. This provides the advantage that the prepared bone replacement paste can be filled, for example using a spatula, more easily into the cavity in the base part.

In another embodiment of the device, venting slots are arranged at the rear end of the filling piston, preferably in a terminal surface of the grip. Elevation of the pressure can be avoided with these venting slots, especially when a piston is fitted on the grip. Furthermore, alternatively or in addition, connecting holes may be arranged for air equilibration between the hollow spaces in the filling piston into which the cannulas of the applicators are inserted.

In another embodiment of the device the filling piston has a longitudinal section with a reduced cross-sectional area. By means of the reduced cross-sectional area, recesses for reducing friction are formed, which leads to a reduction of the force application, especially during insertion of the filling piston into the cavity in the base, filled with bone replacement paste.

Preferably each of the hollow spaces has a diameter of at least 3 mm, preferably of at least 3.5 mm.

In an additional embodiment of the device, each of the hollow spaces has a diameter of at most 8.5 mm, preferably at most 6.5 mm.

In another embodiment of the device the cavity has a volume of at least 0.1 cm³, preferably at least 0.2 cm³.

In yet another embodiment of the device, the cavity has a volume of at most 120 cm³, preferably at most 100 cm³.

In an additional embodiment of the device, the cannula has a front end and is curved forward in the area of the front end at an angle of between 5° and 50°, preferably between 10° and 45°.

In another embodiment of the device the cannula has a front end and in the area of the front end is flexible over a length of between 0-70 mm, preferably 0-60 mm.

In another embodiment the cannula has a lumen that does not have any constrictions at the front end of the cannula, wherein preferably the lumen essentially corresponds to a hollow space of the filling piston.

In a further embodiment of the device the cannula is opaque to x-rays, throughout or only at the front end. This provides the advantage that during application of the bone replacement paste the position of the cannula is visible on an x-ray and the bone replacement paste can be safely and effectively applied. Dense ceramics or metal materials, for example BaSO₄, are often used as the x-ray-impenetrable materials (radio-opacifiers). For example, the BaSO₄ can be added directly to the raw material (e.g., polymer) in the cannula and then extruded.

In another embodiment of the device the cavity is cylindrical or prismatic in shape, preferably has the shape of a plain cylinder.

In an additional embodiment of the device the hollow spaces in the filling piston are cylindrical or prismatic in shape, preferably shaped as plain cylinders.

In an additional embodiment of the device the filling pistons has a front end that is of a flat or concave shape.

In an additional embodiment of the device the concave shaped front end of the filling piston in the area of the second longitudinal axis is made deeper by 0.4 mm to 1.2 mm. This offers the advantage that the bone replacement paste, during insertion of the filling piston into the cavity of the base part, is pressed inward by the front end of the filling piston with its concave design, I.e., against the longitudinal axis, so that the hollow spaces are uniformly filled over their entire cross-section.

In an additional embodiment of the device the first end of the base part is flat, at least in the area of the cavity.

In yet another embodiment of the device, the cavity has a depth T in the direction of the first longitudinal axis and the filling piston has a height H in the direction of the second longitudinal axis, wherein the height H is just as large as or larger than the depth T.

In another embodiment of the device the cannula of the applicator device can be inserted slidably into the open hollow spaces of the filling piston. As a result the cannula can be inserted with a fit having a small amount of play into one of the hollow spaces of the filling piston filled with bone replacement paste, so that the bone replacement paste present in the hollow space is pressed into the lumen of the cannula, pushing the applicator piston toward the rear.

In an additional embodiment of the device, the device also has a spatula, with a spatula blade and a shaft.

Preferably the spatula blade has a base area which is geometrically similar to at least one part of a cross-sectional area of the cavity perpendicular to the first longitudinal axis of the base part. In this way the advantage can be achieved that the spatula blade covers only the part of the cross-sectional area or at the circumference has an adequate play relative to the cavity so that the air enclosed in the bone replacement paste and in the base part can escape during the packing of the bone replacement paste.

In another embodiment of the device the funnel-shaped second end of the base part has at least one rib on the inside. The rib can prevent incorrect insertion or sticking of the filling piston.

In yet another embodiment of the device the funnel-shaped second end of the base part tapers inwardly with a low angle against the cavity. This offers the advantage that the filling piston cannot stick during insertion into the cavity and can be brought into the correct position with a slight rotary movement.

Preferably the applicator does not have an injection needle.

The applicator piston can have a scale defining the intended depth of insertion of the applicator piston into the cannula.

In an additional embodiment of the device, the base part additionally has an outer sleeve that is connected with the second end of the base part by a flange-like broadening of the base part and which extends in the direction of the first longitudinal axis beyond the first end of the base part to a lower end of the sleeve.

In an additional embodiment of the device the filling piston comprises a bonnet-type grip, which extends in the direction of the second longitudinal axis toward the front end over a section of the height H of the filling piston.

In an additional embodiment of the device the first end of the base part comprises a small venting hole in the area of the longitudinal axis.

In yet another embodiment of the device the two or more hollow spaces are non-conical in shape.

Preferably at least the surface of the hollow spaces in the filling piston comprises a material selected from the group of ultra-high molecular weight polyethylenes, poly carbonates, polypropylene, polyamide, polyurethane, polyether ether ketone (PEEK), ionomer, polyvinyl chloride, polyether block amide, styrene ethylene butylene styro or polysulfane. As a result of the good sliding properties of these materials the pressure drop can be further reduced and damage to the particles contained in the bone cement or the bone replacement material can be largely avoided. In addition the bone cement or the bone replacement material is not contaminated due to the slight abrasion of these materials.

The kit according to the invention for application of bone replacement paste comprises a device according to one of the above-described embodiments and with the addition of at least one applicator. This additional applicator gives the advantage that the second or further applicators can be filled with bone replacement paste, for example, even during the intraoperative use of the first applicator.

In a special embodiment the kit also contains at least one additional filling piston. In this way, filling pistons of different designs can be used, e.g., filling pistons with different diameters of the individual hollow spaces, so that cannulas with different diameters can also be used.

In another embodiment of the kit:
a) the hollow spaces of a first filling piston are shaped differently compared to the hollow spaces of a second filling piston;
b) the cannula of a first applicator has an external shape suitable for the hollow spaces of the first filling piston;
c) the cannula of a second applicator has an external shape suitable for the hollow spaces of the second filling piston.

In a special embodiment of the method, prior to filling the base part, the following additional step is performed: mixing the substances required for preparation of the bone replacement paste. In so-called ready-to-use products without application aids, this step is not necessary.

Preferably the mixing is performed by stirring the dry powder mixture with a liquid using the spatula.

In another embodiment of the method, the base part is filled with the prepared bone replacement paste using the spatula.

In another embodiment of the method, before inserting and pressing the filling piston into the cavity of the base part, the following additional step is carried out:
Pressing the bone replacement paste firmly into the cavity of the base part, preferably using the blade of the spatula.

In an additional embodiment of the method, before a cannula is inserted into an applicator device, the filling piston is slightly withdrawn into the cavity of the base part.

In an additional embodiment of the method, the cannula of the applicator is rotated around the axis of the hollow space after inserting it into the hollow space of the filling piston filled with bone replacement paste.

In yet an additional embodiment of the method, the front end of the filling piston is inserted into the cavity up to the first end of the base part.

In the following the invention and further developments of the invention will be explained in greater detail based on the illustrations, some of which are schematic.

These show:

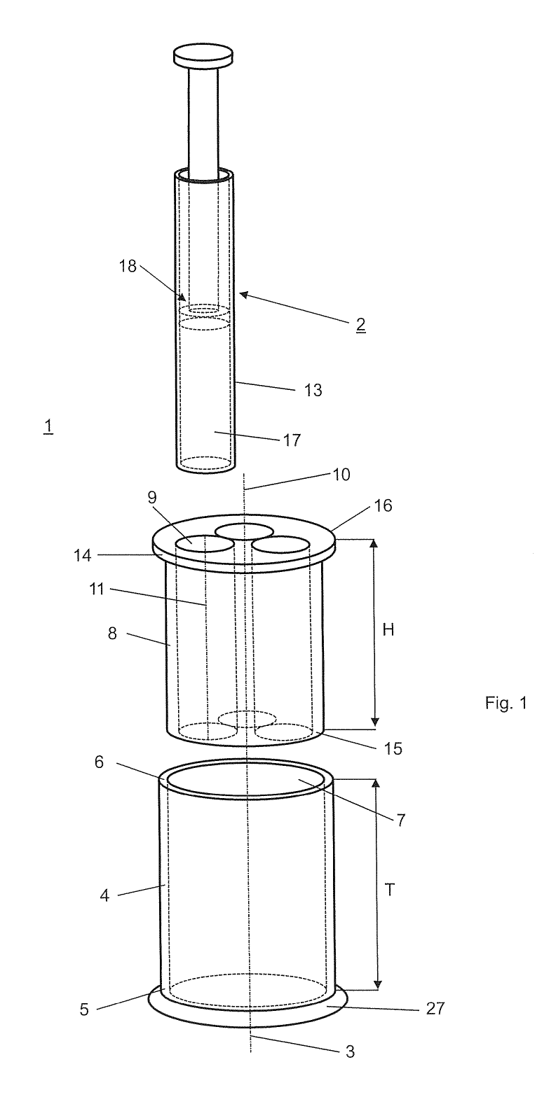

FIG. 1 A perspective view of an embodiment of the device according to the invention;

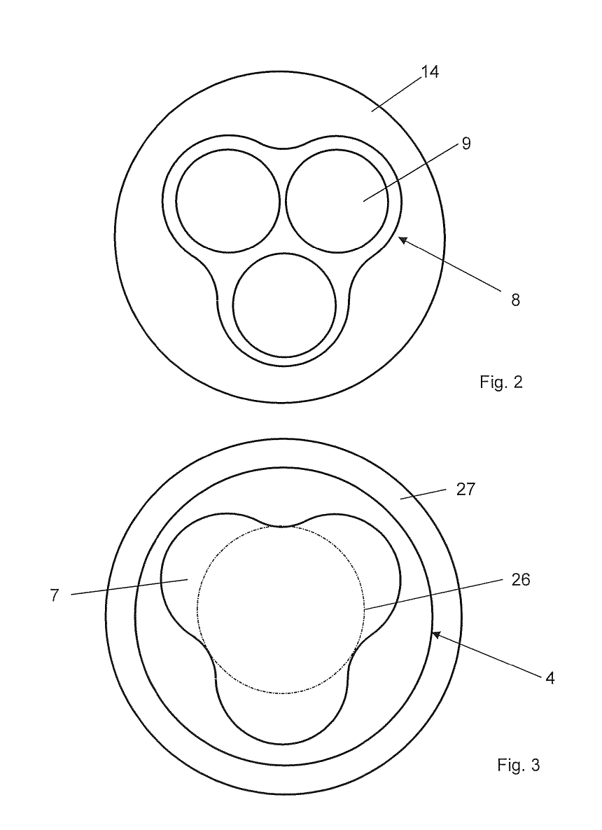

FIG. 2 A top view from the front end to the filling piston of an additional embodiment of the device according to the invention;

FIG. 3 A top view from the second end to the base part of the embodiment of the device according to the invention shown in FIG. 2;

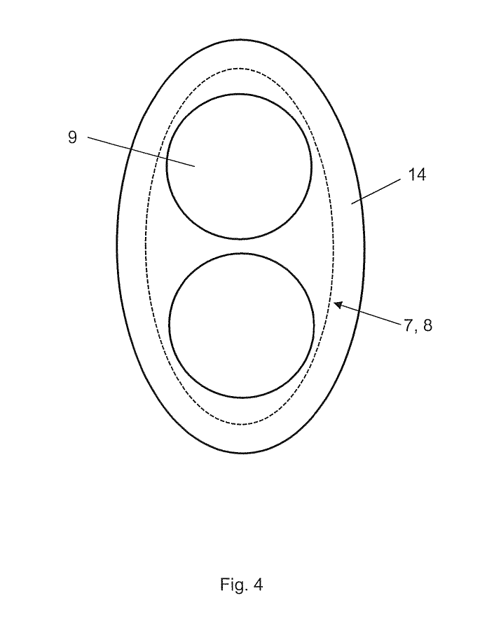

FIG. 4 A plan view of the base part and a filling piston inserted therein of another embodiment of the device according to the invention;

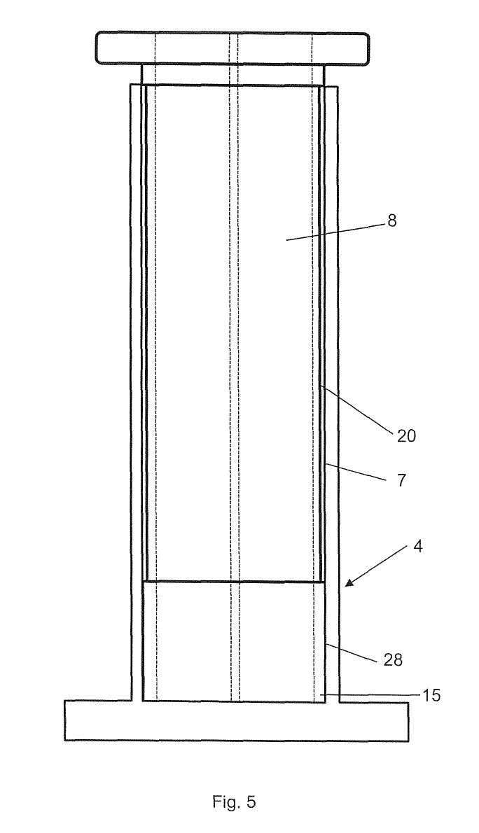

FIG. 5 A longitudinal section through the base part with filling piston inserted according to an additional embodiment of the device according to the invention;

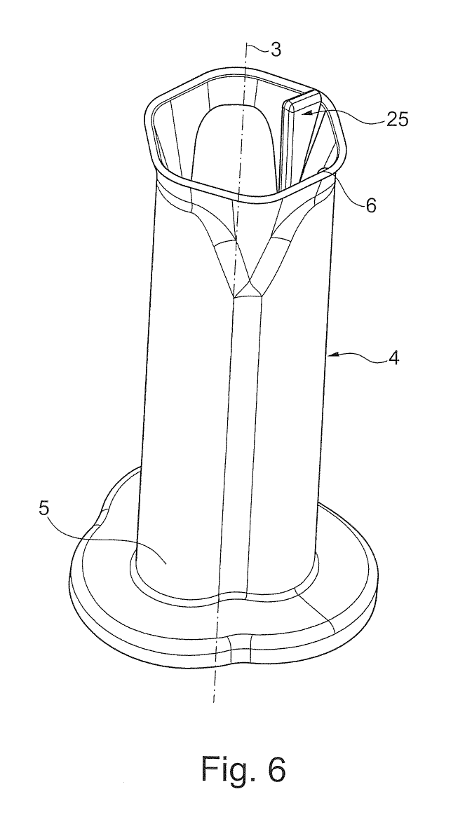

FIG. 6 A perspective view of the base part of another embodiment of the device according to the invention;

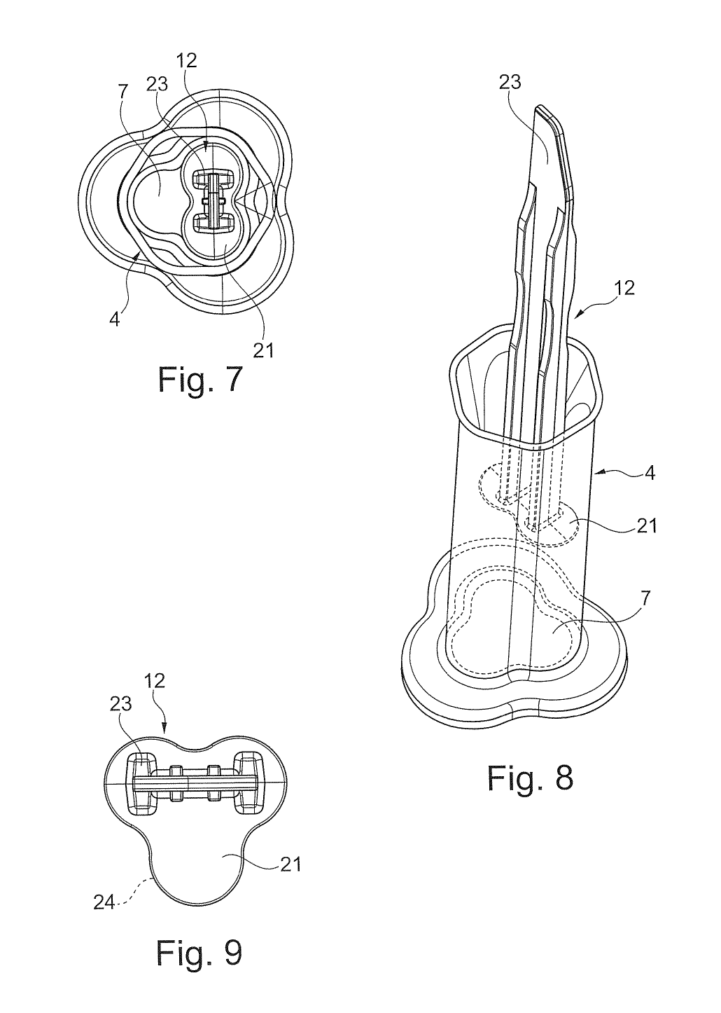

FIG. 7 A plan view of the base part with the spatula inserted according to an additional form of the device according to the invention;

FIG. 8 A schematic representation of the base part with partially inserted spatula of the embodiment of the device according to the invention shown in FIG. 7;

FIG. 9 A plan view of a spatula according to another embodiment of the device according to the invention;

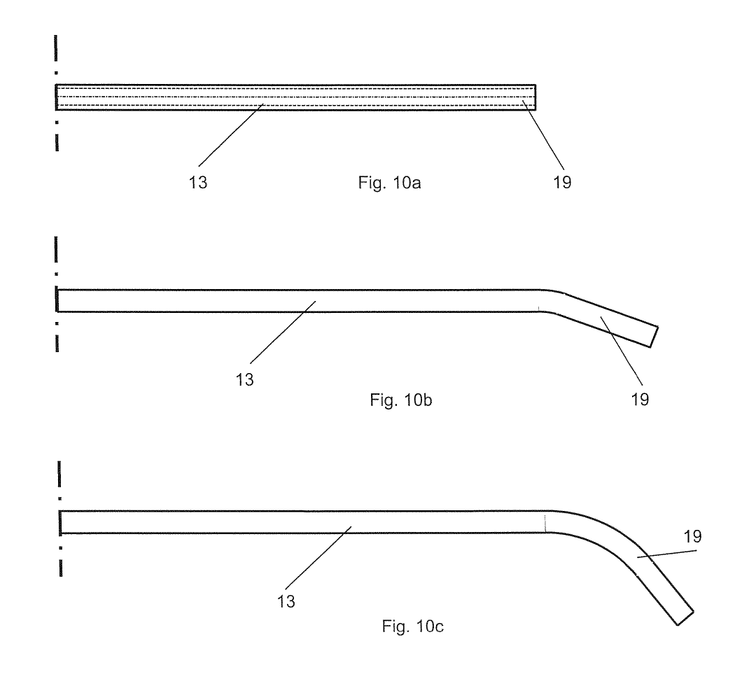

FIGS. 10a-10c Side views of differently shaped cannulas of the applicator devices of another embodiment of the device according to the invention;

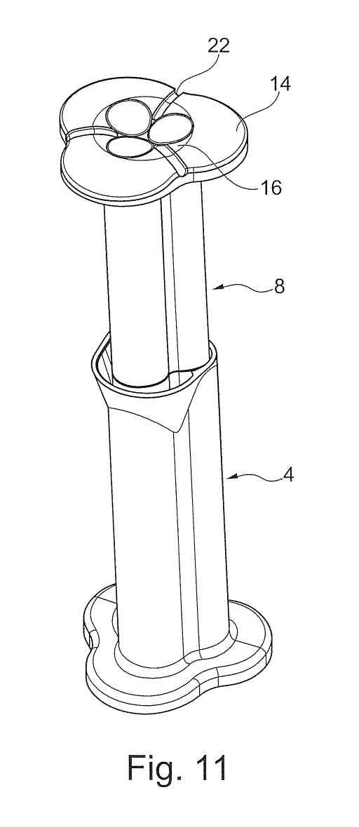

FIG. 11 A perspective view of the base part of the device according to another embodiment of the invention with the filling piston inserted;

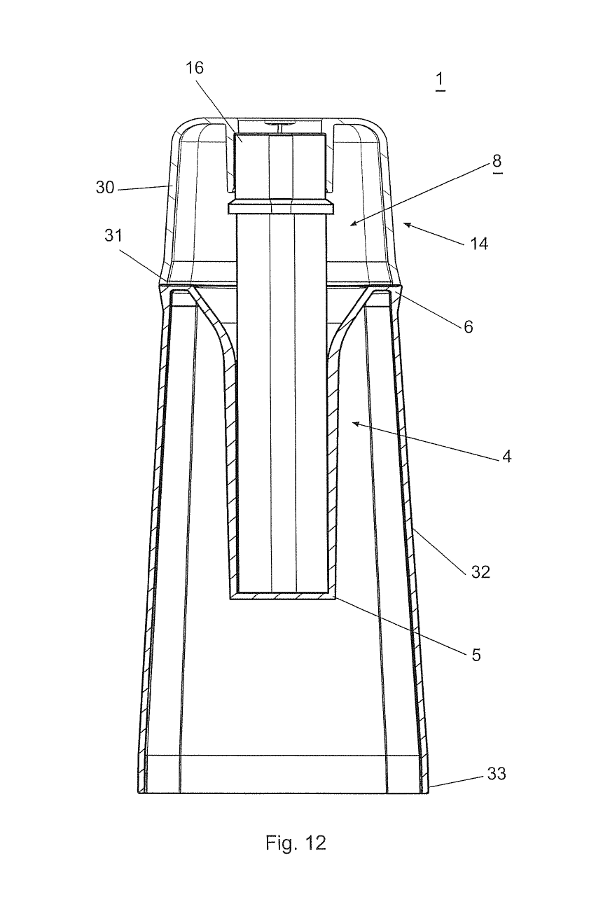

FIG. 12 A longitudinal section through an additional embodiment of the device according to the invention with the filling piston inserted into the base part;

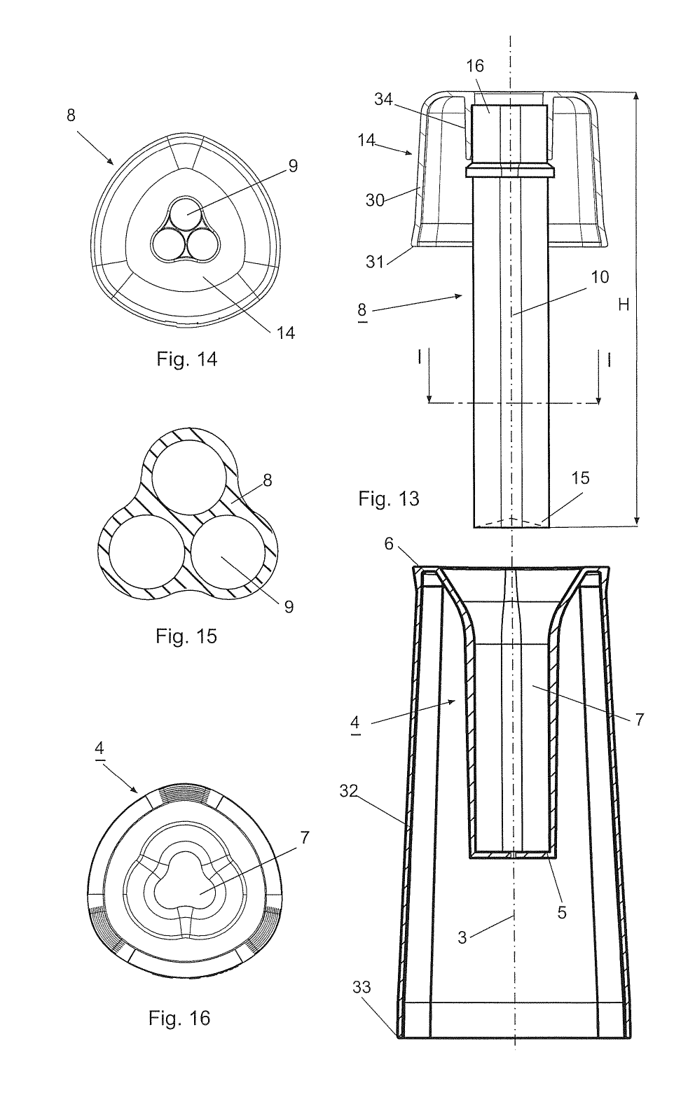

FIG. 13 An exploded view of the embodiment of the device according to the invention shown in FIG. 12;

FIG. 14 A plan view of the filling piston of the embodiment of the device according to the invention shown in FIG. 12;

FIG. 15 A cross-section through the filling piston along line I-I in FIG. 12; and

FIG. 16 A top view of the base part of the embodiment of the device according to the invention shown in FIG. 12.

The embodiment of the device 1 according to the invention shown in FIG. 1 comprises a base part 4 fillable with a bone replacement paste, having a cavity 7, a filling piston 8 insertable into the cavity 7, having three hollow spaces 9, and an applicator device 2 having a cannula 13 that can optionally be inserted into one of the three hollow spaces 9. The base part 4 is designed like a container and comprises a first end 5, a second end 6 and a cavity 7 which defines a first longitudinal axis 3. The cavity 7 is closed at the first end 5 of the base part 4 and open at the second end 6. The filling piston 8 that can be inserted slidably into the cavity 7 comprises a second longitudinal axis 10 and, for example, three plain-cylindrical hollow spaces 9, which penetrate the filling piston 8 in parallel to the second longitudinal axis 10. The hollow spaces 9 have hollow space axes 11 parallel to the second longitudinal axis 10, arranged in the corners of an equilateral triangle positioned perpendicular to the second longitudinal axis 10. The cavity 7 in the base part 4 is designed as a plain cylinder and the filling piston 8 is likewise designed as a plain cylinder fitting with this on the outside. The filling piston 8 can be slidably inserted into the cavity 7 in the base part 4 and has a small amount of play in the cavity 7, which is suitable for reducing the resistance when inserting the filling piston 8 but nevertheless allows penetration of bone replacement paste into the interval caused by the play between the outer wall of the filling piston 8 and the inner wall of the cavity 7. Alternatively the cavity 7 and the filling piston 8 may be prismatic in design.

In addition, in an alternative embodiment the hollow spaces 9 in the filling piston 8 may be prismatic in form and for example have a polygonal (e.g., triangular, rectangular, trapezoidal or rhomboidal) cross-section. The cavity 7 has a depth T measured in the direction of the first longitudinal axis 3, and the filling piston 8 measured in the direction of the second longitudinal axis 10 has a height H, where the height H is greater than the depth T. At the first end 5 of the base part 4, a blade 27 is arranged, which perpendicular to the first longitudinal axis 3 has a larger cross-sectional area than the next higher section of the base part 4 and defines a support surface. Furthermore at the rear end 16 of the filling piston 8 a grip 14 is arranged. The applicator device 2 comprises a cannula 13 with a lumen 17 and an applicator piston 18 that can be inserted into the lumen 17 of the cannula 13. Optionally the cannula 13 may be slidably inserted into one of the three hollow spaces.

In FIGS. 2 and 3 an additional embodiment of the device 1 according to the invention is shown, the base part 4 and filling piston 8 of which differ from the embodiments shown in FIG. 1 in that the cavity 7 in the base part 4 in a cross-sectional surface perpendicular to the first longitudinal axis 3 is formed in a cloverleaf shape and the filling piston 8, fitting it on the outside, is likewise formed in a cloverleaf shape in a cross-sectional surface perpendicular to the second longitudinal axis 10. The filling piston 8 is slidably insertable into the cavity 7 in the base part 4 and also has slight play in the cavity 7, which is suitable for reducing the resistance during insertion of the filling piston 8 without allowing penetration of bone replacement paste into the intermediate space caused by the play between the outer wall of the filling piston 8 and the inner wall of the cavity.

The filling piston 8 likewise comprises three plain-cylindrical hollow spaces 9, which penetrate the filling piston 8 in parallel to the second longitudinal axis 10. The hollow spaces 9 point toward hollow space axis 11 parallel to the second longitudinal axis 10 and placed in the corners of an equilateral triangle positioned perpendicular to the second longitudinal axis 10. The outer shape of the filling piston 8 is formed such that around each hollow space 9—viewed in cross-section—a lug with a periphery in the shape of a circular arc is formed, so that the hollow space axis 11 of each circular cylindrical hollow space 9 is arranged in the center of the circular arc-shaped periphery of a lug. Here the diameters of the circular arc-shaped periphery of the lugs and the hollow spaces are selected such that the wall strengths of the outer and intermediate walls guarantee adequate stability of the device 1. As is apparent in FIG. 3, the three lugs are arranged around a circular area 26 and distributed uniformly over the circumference of this central circular area 26. The transitions between the lugs and the central circular area 26 are rounded in a cross-sectional area perpendicular to the first longitudinal axis 3, so that in parallel to the first longitudinal axis 3, constrictions are formed in the cavity 7. Fitting with this, the transitions between the lugs on the filling piston 8 are rounded, so that in parallel to the second longitudinal axis 10, concave channels are formed on the filling piston 8. This design of the cross-sectional areas of the cavity 7 and the filling piston 8 increases the ratio between the sum of all cross-sectional areas of the hollow spaces 9 and the cross-sectional area of the cavity 7, so that the pressure drop that occurs when the filling piston 8 is pressed into the cavity 7 filled with the bone replacement paste is also reduced considerably by the change in cross-section between the cavity 7 and the hollow spaces 9 compared with the embodiment shown in FIG. 1 with circular cross-sectional area of the cavity 7 and the filling piston 8.

In FIG. 4 an additional embodiment of the device 1 according to the invention (without the applicator device 2) is shown, which differs from the embodiment shown in FIGS. 1 and 2 in that the cavity 7 in the base part 4 and the filling piston 8 perpendicular to the first and second longitudinal axes 3;10 each have an elliptical cross-sectional area, and that in the filling piston 8, two hollow spaces 9 are arranged, wherein the connecting line between the hollow space axes 11 perpendicular to the second longitudinal axis 10 lies on the longitudinal axis of the elliptical cross-sectional areas of the filling piston 8 and the cavity 7. Alternatively, the cross-sectional areas of the cavity 7 and the filling piston 8 perpendicular to the first and second longitudinal axes 3;10, respectively, may be of oval design.

In the following, examples of dimensions of the cavity 7 in the base part 4 and the hollow spaces 9 in the filling piston 8 are shown for two different embodiments of the device 1 according to the invention.

### First Illustrative Embodiment

The device 1 comprises a cavity 7 of circular cylindrical form in the base part 4 and three hollow spaces 9 in the filling piston 8 (FIG. 1). The hollow spaces 9 in the filling piston 8 each have a diameter of 5.3 mm and thus each have a cross-sectional area q of 22.1 mm², while the cavity 7 in the base part 4 can have a diameter of 14 mm and thus a cross-sectional area Q of 154 mm². For a volume V of the cavity 7 of 10 cm³, the cavity 7 has a depth T of at least 65 mm. For a volume v of a hollow space 9 of 3.33 cm³, a height H of the filling piston 8 of at least 150.7 mm results. The ratio between the volume v of a hollow space 9 and the volume V of the cavity 7 then amounts to 0.33. The ratio between the total of the cross-sectional areas q of the hollow spaces 9 and the cross-sectional area Q of the cavity 7 in this case is 0.43.

The diameter of the lumen 17 in the cannula 13 of the applicator 2 and thus also the minimum length of the cannula 13 depends on the wall thickness of the cannula 13. Additional examples of dimensions are:
the hollow spaces 9 can have lengths between 50 and 150 mm for diameters between 3 and 10 mm and lengths between 50 and 100 mm for diameters between 4 and 10 mm;
the cavity 7 can have lengths between 30 and 100 mm for diameters between 5 and 50 mm and lengths between 40 and 80 mm for diameters between 10 and 20 mm; and
the cannula 13 can have lengths between 50 and 300 mm for lumen diameters between 3 and 10 mm and lengths between 100 and 200 mm for lumen diameters between 4 and 10 mm.

Typically the lumen 17 has a diameter of 5.2 mm and the cannula 13 a length of 200 mm.

### Second Illustrative Embodiment

A most compact arrangement of the hollow spaces 9 in the filling piston 8 can be achieved with 7 hollow spaces 9 wherein a central hollow space 9 is surrounded by 6 additional hollow spaces 9, the hollow space axes 11 of which—in a cross-sectional area of the filling piston 8 perpendicular to the longitudinal axis—are arranged in the corners of a hexagon, wherein the diameter of the cavity 7 amounts to little more than three times the diameter of a hollow space 9. The hollow spaces 9 in the filling piston 8 each have a diameter of 8 mm and thus each have a cross-sectional area q of 50.25 mm², while the cavity 7 in the base part 4 may have a diameter of 26 mm and thus a cross-sectional area Q of 531 mm². For a volume V of the cavity 7 of 24.5 cm³, the cavity 7 has a depth T of at least 46 mm. For a volume v of a hollow space 9 of 3.5 cm³, a height H of the filling piston 8 of at least 70 mm results. The ratio between the volume v of a hollow space 9 and the volume V of the cavity 7 then amounts to 0.14. In the case of such a tight arrangement a ratio between the sum of the cross-sectional areas q of the hollow spaces 9 and the cross-sectional area Q of cavity 7 of 0.66 is obtained.

In FIG. 5 an embodiment of the filling piston 8 is shown, which is characterized in that the filling piston 8 at its front end 15 has a first longitudinal section 28 with a cross-sectional area (as described above) corresponding to the cross-sectional area of the cavity 7, and following this first longitudinal section 28 has a second longitudinal section 20 with a reduced cross-sectional area, so that the frictional resistance is reduced when inserting the filling piston 8 into the cavity 7.

In FIG. 6 an embodiment of the base part 4 is shown, which is characterized in that the second end 6 of the base part 4 is designed as a funnel, wherein the funnel-like second end 6 tapers inwardly with a low slope toward the cavity 7, so that the filling piston cannot stick during insertion into the cavity and can be brought into the correct position with a slight rotary movement. In addition, on the inside of the funnel-shaped second end 6 of the base part 4 at least one rib 25 is arranged, which extends in parallel to the first longitudinal axis 3, so that incorrect insertion of the filling piston can be prevented.

FIGS. 7 and 8 show an embodiment of a spatula 12 that can be used with the device 1 according to the invention shown in FIGS. 2 and 3, which serves for filling the base part 4 with the prepared bone replacement paste and for pressing the bone replacement paste into the cavity 7 of the base part 4. The spatula 12 comprises a spatula blade 21 and a shaft 23. The spatula blade 21 in this embodiment covers only part of the cross-sectional area of the cavity 7 and perpendicular to the shaft 23 has a base surface, which essentially has the shape of a horizontal number eight. The two rounds of the eight correspond to two adjacent lobes of the cloverleaf-shaped cross-sectional surface of the cavity 7, so that the spatula blade 21 can be moved in a chosen orientation into the cavity 7. Therefore air enclosed in the bone replacement paste and in the base part 4 can escape when the bone replacement paste is being pressed through the channel, the cross-section of which is formed by the third lobe of the cloverleaf-shaped cross-sectional area of the cavity 7.

In FIG. 9 an additional embodiment of a spatula 12 that can be used with the device of the invention 1 according to FIGS. 2 and 3 is shown. In the embodiment shown here the spatula blade 21 has a base area 24 that is similar to the cross-sectional area of the cavity 7 perpendicular to the first longitudinal axis of the base part 4, so that the spatula blade 21 has play at the circumference vis-à-vis the cavity 7. In this case the air enclosed in the bone replacement paste and in the base part can escape when the bone replacement paste is pressed firmly through the intermediate space formed by the play between the spatula blade 21 and the inner wall of the cavity 7.

FIGS. 10a to 10c show different embodiments of the cannulas 13, which:
may be formed as straight cannulas 13 (FIG. 10a);
at the front end 19 of the cannula 13 can be bent forward at an angle of between 5° and 50°, preferably between 10° and 45° (FIG. 10b); or
at the front end 19 of the cannula 13 may be flexible over a length of between 0-70 mm, preferably 0-60 mm, so that the cannulas can be bent intraoperatively (FIG. 10c);
the cannulas 13 may be transparent to x-rays (without a radio-opacifier); or
the cannulas 13 may have an x-ray marker at the anterior end (contact with defect); or
the cannulas 13 may be impermeable to x-rays over the entire cannula volume (e.g., through addition of BaSO₄ to the raw material of the cannulas).

FIG. 11 shows an additional embodiment of the device 1 according to the invention, which differs from the embodiment shown in FIGS. 2 and 3 only in that venting slots 22 are arranged at the rear end 16 of the filling piston 8 in the terminal surface of the grip 14, extending from a central indentation between the hollow spaces 9 at the rear end 16 of the filling piston 8 toward the outside, so that when the filling piston 8 is pressed into the cavity 7 air can escape from the hollow spaces 9 by way of an object covering the rear end 16 of the filling piston 8. Furthermore, connections (holes or grooves, not shown) can be arranged in the hollow spaces 9 in the filling piston 8, into which the cannula 13 of the applicator 2 is inserted, to provide for air equilibration between the hollow spaces.

An additional embodiment of the device 1 according to the invention is shown in FIGS. 12 to 16; this differs from the embodiment shown in FIGS. 2 and 3 only in that the base part 4, instead of the flange-like blade 27 arranged at the first end 5, is equipped with an outer sleeve 42 and the filling piston 8 has a bonnet-like grip 14, which forms a cap 30 extending in the direction of the second longitudinal axis 10 toward the front end 15 in a section of the height H of the filling piston 8. The outer sleeve 32 is connected to the second end 6 of the base part 4 by a flange-like expansion of the base part 4 and expands in the direction of the first longitudinal axis 3 over the first end 5 of the base part 4 to the lower end 33 of the sleeve 32.

The filling piston 8 is welded at its lower end 16 by ultrasound into a recess 34 in the cap 30. The cap 30 and the filling piston 8 form a unit for the user. Alternatively the connection between the back end 16 of the filling piston 8 and the receiver 34 in the cap 30 can also be designed as an irreversible press fit connection or an irreversible snap connection. In other embodiments the cap 30 and the filling piston 8 can also be made of one piece.

The cap 30 and the sleeve 32 are designed such that the outer shape of the cap 30 expands from the back end 16 of the filling piston 8 toward the lower end 31 of the cap 30 and the external shape of the sleeve 32 expands from the second end 6 of the base part 4 toward the lower end 33 of the sleeve 32.

The cap 30 and the sleeve 32, in an illustrative and non-limiting example, each have a cross-sectional area orthogonal to the first longitudinal axis 3 and the second longitudinal axis 10, having the shape of a bulging triangle similar to a Reuleaux triangle and a contour made up of three flattened circular arcs with rounded corners. This shape makes it possible for the cap 30 and/or the sleeve 32 on one hand to be ergonomically suited for manual use and on the other hand not to roll away if one of the parts or the assembled device 1 falls on its side surface.

When the filling piston 8 is inserted completely into the cavity 7 of the base part 4, the lower end 31 of the cap 30 lies on the second end 6 of the base part 4. The edges at the lower end 31 of the cap 30 and at the second end 6 of the cap 4 are rounded so as not to damage the user's glove. The cap 30 and the sleeve 32 have a slight widening to prevent slippage of the hand. The sleeve 32 also has a 3-dimensional structuring on its outer surface to prevent slipping of the glove.

The front end 15 of the filling piston 8 is shown, in an illustrative and a non-limiting example, as concave, wherein the concave shaped front end 15 of the filling piston 8 is indented by 0.4 mm to 1.2 mm in the area of the longitudinal axis 10. Alternatively the front end 15 of the filling piston 8 can also be made flat. Furthermore the first end 5 of the base part 4 has a small venting hole 29 in the area of the first longitudinal axis 3.

In analogy to the embodiment shown in FIG. 4, the second end 6 of the base part 4 may be formed in the shape of a funnel, wherein the second end 6 formed like a funnel tapers with a low slope toward the cavity 7, so that the filling piston cannot stick during insertion into the cavity and can be brought into the correct position with a slight rotary movement.

### Intraoperative Preparation of the Bone Replacement Paste and Filling of the Applicator Device (FIGS. 1 and 5 to 9)

1) Mixing the materials required for preparation of the bone replacement paste.
   The dry powder mixture is stirred with liquid using a spatula 12. This step is omitted in the case of ready-to-use products without application aids;
2) Filling the base part 4 with the prepared bone replacement paste.
   The prepared bone replacement paste is filled into the bottom part of the loading unit using spatula 12 (preferably using the funnel function);
3) Pressing the bone replacement paste firmly into the cavity 7 of the base part 4.
   The spatula blade 21 can be used to “tamp down” the compound into the base part 4;
4) Inserting and pressing the filling piston 8 into the cavity 7 of the base part 4, so that the hollow spaces 9 of the filling piston 8 are filled with bone replacement paste;
   As soon as all of the compound has been filled into the base part, the filling piston with the 3 hollow cylinders is manually pressed onto the base part. In this way the hollow spaces of the filling piston are filled. In such cases the cross sectional areas are likewise optimized such that the smallest possible application of force results.
5) Inserting the cannula 13 of an applicator device 2 into one of the hollow spaces 9 of the filling piston 8 filled with bone replacement paste so that the bone replacement paste present in the hollow space 9 of the filling piston 8 is advanced into the lumen 17 of the cannula 13.
   Preferably in this process the filling piston 8 is pressed against the base part 4 using the gripping element 14 so that the filling piston 8 is not raised by the pressure on the bone replacement paste in the cavity 7 of the base part 4. The bone replacement paste can be pushed off by slightly rotating the applicator device 2.
6) Introducing the bone replacement paste into the bone defect using the applicator device 2.

Steps 5) and 6) can be repeated as needed until the entire bone defect is filled with bone replacement paste.

One embodiment of the kit in accordance with the invention comprises, in addition to a device 1 according to one of the embodiments shown in FIGS. 1 to 10, at least one additional applicator device 2 which makes it possible that, for example, even during the intraoperative use of the first applicator device 2, one or more additional applicator devices 2 can be filled with bone replacement paste. In additional embodiments of the kit according to the invention, this may also comprise one or more additional filling pistons 8, so that differently shaped filling pistons 8 may be used, e.g., filling pistons 8 each with different diameters of the hollow spaces 9, so that cannulas 13 with different diameters may also be used. Alternatively, the hollow spaces 9 of a first filling piston 8 may be shaped differently from the hollow spaces 9 of a second filling piston 8 (e.g., plain cylindrical hollow spaces 9 versus prismatic hollow spaces 9), wherein at least the cannula 13 of a first application device 2 has an external shape (e.g., plain cylindrical) conforming to the hollow spaces 9 of the first filling piston 8 and the cannula 13 of a second applicator device 2 has an external shape conforming to the hollow spaces 9 of the second filling piston 9 (e.g., prismatic).

Although, as described in the preceding, various embodiments of the present invention exist, these are to be understood such that the various features can be used both individually and in any desired combination.

Therefore this invention is not simply limited to the above-named, particularly preferred embodiments.

1. A device for applying bone replacement pastes or bone cements comprising:
A) a container-like base part having a first end, a second end and a cavity, which defines a first longitudinal axis, is closed at the first end and is open at the second end; and
B) a filling piston that is slidably insertable into the cavity, which has a second longitudinal axis, and which has two or more hollow spaces that extend through the filling piston coaxially or parallel to the second longitudinal axis.

2. The device according to claim 1, wherein the device further comprises an applicator device, which has a cannula with a lumen, and an applicator piston that is configured to be inserted in the lumen of the cannula.

3. The device according to claim 1, wherein the cavity has a cross-sectional area Q perpendicular to the first longitudinal axis, each hollow space has a cross-sectional area q perpendicular to the second longitudinal axis, and a ratio between a sum of the cross-sectional areas q and the cross-sectional area Q is between 0.25 and 0.95.

4. The device according to claim 1, wherein the filling piston comprises more than two hollow spaces, which penetrate the filling piston coaxially or in parallel to the second longitudinal axis.

5. The device according to claim 1, wherein each of the hollow spaces has a volume v, wherein the cavity has a volume V, and wherein a ratio between the volume v and the volume V of the cavity amounts to at least 0.05.

6. The device according to claim 1, wherein the hollow spaces each have hollow space axes parallel to the second longitudinal axis, which are arranged in angles of a regular polygon located perpendicular to the second longitudinal axis.

7. The device according to claim 1, wherein the cavity perpendicular to the first longitudinal axis and the filling piston on an outside perpendicular to the second longitudinal axis have a non-circular cross-sectional area.

8. The device according to claim 1, wherein a grip is attached to the filling piston.

9. The device according to claim 1, wherein the base part is made in a funnel shape at the second end.

10. The device according to claim 1, wherein venting slots are arranged at a rear end of the filling piston.

11. The device according to claim 1, wherein the filling piston has a longitudinal section with a reduced cross-sectional area.

12. The device according to claim 1, wherein each of the hollow spaces has a diameter of at least 3 mm.

13. The device according to claim 1, wherein each of the hollow spaces has a diameter of at most 8.5 mm.

14-15. (canceled)

16. The device according to claim 2, wherein the cannula has a front end and is curved forward in an area of the front end at an angle of between 5° and 50°.

17. The device according to claim 2, wherein the cannula has a front end and wherein an area of the front end is flexible over a length of greater than 0 mm up to 70 mm.

18. The device according to claim 2, wherein the cannula has a lumen that does not have any constrictions at a front end of the cannula.

19. The device according to claim 2, wherein the cannula is opaque to x-rays, throughout or only at a front end.

20-21. (canceled)

22. The device according to claim 1, wherein the hollow spaces in the filling piston have a front end that is of a flat or concave shape.

23. The device according to claim 1, wherein the hollow spaces in the filling piston have a front end that is of a concave shape, which is deeper by 0.4 mm to 1.20 mm in an area of the second longitudinal axis.

24. (canceled)

25. The device according to claim 1, wherein the cavity has a depth T in a direction of the first longitudinal axis, wherein the filling piston has a height H in a direction of the second longitudinal axis, and wherein the height H is just as large as or larger than the depth T.

26. The device according to claim 2, wherein the cannula of the applicator device is configured to be slidably insertable into the hollow spaces of the filling piston.

27. The device according to claim 1, wherein the device further comprises a spatula including a spatula blade and a shaft.

28. The device according to claim 27, wherein the spatula blade has a base area which is geometrically similar to at least one part of a cross-sectional area of the cavity perpendicular to the first longitudinal axis of the base part.

29. The device according to claim 27, wherein the spatula blade has play relative to the cavity at the circumference.

30. The device according to claim 9, wherein the funnel-shaped second end of the base part has at least one rib on an inside.

31. The device according to claim 9, wherein the funnel-shaped second end of the base part tapers inwardly with a low angle against the cavity.

32. The device according to claim 2, wherein the applicator device does not comprise injection needles.

33. The device according to claim 1, wherein the base part further includes an outer sleeve that is connected with the second end of the base part by a flange-like broadening of the base part and which extends in a direction of the first longitudinal axis beyond the first end of the base part to a lower end of the sleeve.

34. The device according to claim 1, wherein the filling piston has a bonnet-type grip, which extends in a direction of the second longitudinal axis toward a front end over a section of a height H of the filling piston.

35. The device according to claim 1, wherein the first end of the base part has a small venting hole in an area of the longitudinal axis.

36. The device according to claim 1, wherein the two or more hollow spaces are non-conical in shape.

37. A kit for application of bone replacement pastes comprising a device according to claim 1 and, additionally, at least one more applicator device.

38. The kit according to claim 37, wherein the kit further comprises at least one filling piston.

39. The kit according to claim 38, wherein:
a) the hollow spaces of a first filling piston are shaped differently as compared to the hollow spaces of a second filling piston;
b) a cannula of a first applicator device has an external shape conforming to the hollow spaces of the first filling piston; and
c) a cannula of a second applicator device has an external shape conforming to the hollow spaces of the second filling piston.

40-47. (canceled)
